(19) United States
(12) Patent Application Publication
(10) Pub. No.: US 2006/0140863 A1
(43) Pub. Date: Jun. 29, 2006

(54) Production of a dye agent for colouring cells in the human or animal body

(75) Inventors: Hasso Meinert, Neu-Ulm (DE); Bernard Gunther, Dossenheim (DE); Yong-Keun Kim, Neu-Ulm (DE); Wilfried Hiebl, Illertissen (DE)

Correspondence Address: CROWELL & MORING LLP;INTELLECTUAL PROPERTY GROUP, P.O. BOX 14300, WASHINGTON, DC, 20044-4300, US

(73) Assignee: Fluoron GMBH, Neu-Ulm (DE)

(21) Appl. No.: 10/531,293

(22) PCT Filed: Oct. 10, 2003
(86) PCT No.: PCT/EP03/11251

§ 371 Date: Nov. 17, 2005

(30) Foreign Application Priority Data
Oct. 14, 2002 (DE) 102 47 781.7
Nov. 28, 2002 (DE) 102 55 601.6

(51) Int. Cl. A61K 49/00; G01N 33/48

(52) U.S. Cl. 424/9.6; 435/40.5

(57) ABSTRACT

Use of a biocompatible dye, for example patent blue V or brilliant blue R, for the production of a dye agent for non-cytotoxic visualisation of cells, in particular delimiting or separating membranes in the human or animal body, in particular in the eye.

### FIELD OF THE INVENTION

The present invention concerns the production of a dye agent for coloring cells in the human or animal body.

### BACKGROUND OF THE INVENTION

For that purpose it is known from WO 99/58160 to use trypan blue as a dyestuff. That compound which is known from the class of diazo dyes is used in an aqueous solution for staining the anterior capsule for a cataract operation on the eye. By virtue of visualization of the anterior capsule, the surgeon can recognize the outline of capsulorhexis, whereby phacoemulsification is facilitated.

Trypan blue is a cytotoxic substance, as is known for example from Solomon K D et al: Protective effect of the anterior lens capsule during extra capsular cataract extraction, OPHTHALMOLOGY, Vol 96, No 5, May 1989, 591-597, and Veckener M et al: Ocular toxicity study of trypan blue injected into the vitreous cavity of rabbit eyes, Graefe's Arch. Clin. Ex. Ophthalmol. (2002) 239:698-704. When using trypan blue therefore complete flushing out in particular of the region of the eye in which the trypan blue was used as a dye agent is required immediately after the cataract operation in order to prevent it from remaining in the body or in the eye for a prolonged period of time.

It is known from U.S. Pat. No. 4,764,360 to add to a high molecular polymer which forms a carrier, a dye of a molecular weight of at least 10,000. That is intended to prevent the dye from penetrating into the surrounding body tissue. The dye is intended only to stain the high molecular carrier.

It is also known (E Kutchera, ‘Vitalfäirbung der abgehobenen Netzhaut und ihre Defekte’, Albrecht v. Graefes Arch klin exp Ophthal 178, 72-87 (1969)) for the dye patent blue to be intra-vitreally injected to render visible defects involving the entire retina, in the case of retina detachment. A 0.47% patent blue hyaluronic acid solution was used for the intra vitreal injection. Visualisation of retina detachment is extremely time-consuming and takes place only some days after the injection.

### DESCRIPTION OF THE INVENTION.

The object of the invention is to provide the production of a dye agent with a lack of cytotoxicity, which is suitable for rendering visible membranes with a delimiting or separating function or membranes which have occurred due to disease in the human or animal body.

According to the invention that object is attained by the use of an aqueous physiologically compatible solution in which a dye which does not represent a vital dye and is biocompatible is dissolved, for the production of a dye agent for coloring cells in the human or animal body. The appendant claims set forth advantageous developments of the invention.

In the case of the invention, the use of a biocompatible dye which does not represent a vital dye, without a carrier, in a physiologically compatible aqueous solution of in particular sodium chloride which can be adjusted with a buffer to a pH of between 6.8 and 7.8, in particular about 7.4, provides a dye agent for coloring cells, in particular separating or delimiting membranes, in the human and animal body. The coloring agent is a non-polymeric, low molecular, water soluble dye. The coloring agent used in the invention can be used for vitality testing. Unlike conventional vitality dyes, the biocompatible dye used in the invention can also color the living cells in addition to the dead cells, and also distinguish the dead cells from the living material.

Preferably a triphenylmethane dye is used as a water soluble, low-molecular dye. The dye is used in a carrier free condition. Examples of such suitable dyes are patent blue and brilliant blue R, the latter being known from protein staining in gel electrophoresis.

Patent blue is preferably a patent blue V which is allowed as a foodstuff dye (L blue 3=E 131) (C54H62CaN4014S4. MG: 1159, 45).

The buffer used can be a phosphate, hydrogen carbonate or citrate buffer, the pH value of which can be adjusted by means of sodium hydroxide. The concentration of the biocompatible dye, for example patent blue V, in aqueous solution, is preferably between 0.6 and 2.5 g/l, in particular about 1.2 g/l. Spontaneous staining of the desired regions in the human or animal body is achieved.

The dye agent can be used for coloring the lens capsule, in particular the anterior capsule, in a cataract operation. Staining is effected prior to capsulorhexis and phacoemulsification.

For the staining operation, the aqueous humor is sucked away through a corneal or scleral tunnel incision and the anterior chamber is then filled with a gas, in particular air. About 0.3 ml of dye agent solution, for example patent blue V, is administered into the anterior chamber with a cannula. This causes staining of the lens capsule which is delimited by the pupil edge of the iris. After some seconds the anterior chamber is flushed out with a sodium chloride solution to wash out the dye which is not required.

Then a viscoelastic solution is introduced into the anterior chamber of the eye for carrying out the cataract operation in the usual manner. By virtue of the blue coloration of the anterior capsule the outline of capsulorhexis is clearly apparent and can be clearly distinguished from the gray tissue of the lens core.

In addition the dye agent can be used for coloring the Membrana limitans interna or for example membranes which have occurred as a consequence of PVR (proliferative vitreoretinopathy), in particular epiretinal membranes on the retina or at the rear surface of the vitreous humor delimitation membrane, in particular in relation to retina and vitreous humor surgery.

When removing for example an epiretinal membrane from the retina the dye, for example patent blue V, is selectively applied to the membrane to be removed in about 0.3 ml of the specified buffer solution, by means of a cannula which is introduced by way of the Pars plana. The vitreous humor can be previously replaced entirely or partially by a gas filling, as is used in the usual manner in vitreous humor or retina surgery, in particular macula surgery. When staining the epiretinal membrane, staining of the adjacent retina tissue can possibly take place, with a lesser degree of coloration. Upon removal of the membrane from the subjacent, non colored retina tissue, then gives a good contrast. After the staining operation excess dye agent solution is flushed out and the free space filled by the above mentioned gaseous vitreous humor substitute. By virtue of the coloring action, it is possible to operate with an instrument which is not lit or which has only weak lighting, when removing the membrane. That considerably reduces light toxicity when there is sufficient contrast perception. Particularly in the case of use in connection with epiretinal membranes (epiretinal neuroglia, macular pucker, surface wrinkling), the use of the dye agent solution forms a valuable aid in looking for and removing the membranes.

If in the case of a macula foramen with an increasing hole size, removal of the Membrana limitans interna is required, coloring of that membrane with the dye agent solution is found to be an advantageous aid in looking for and removing that membrane during vitreous humor surgery.

In addition it is possible for a viscoelastic material, for example hyaluronic acid, which is used as an aid in ophthalmological surgery, to be colored with the aqueous dye agent solution. In particular that makes it possible in a cataract operation to achieve an improvement in the contrast of the viscoelastic agent with respect to the intraocular tissue, in particular the iris of the eye and the fundus reflex.

In comparison with the conventional trypan blue which has a teratogenic or mutagenic action (Cahen RL: Evaluation of the teratogenicity of drugs, Clin. Pharmacol. Ther., 1964, 5, 480-514 and Produktinformation BLURHEX™, Dr Agarwal's Pharma Ltd, Chennai (India)), the biocompatible solution according to the invention, for example of patent blue V or brilliant blue R, does not have any cytotoxicity.

To demonstrate lack of cytotoxicity, mouse cells L 929 and ARPE-19-cells were treated with the dye agent according to the invention patent blue V with differing levels of concentration over between 68 and 72 hours in an incubator. The vitality of the cells and a deduced cytotoxicity is quantitatively determined by determining the protein content of the treated cell cultures in comparison with untreated control cultures. The protein content is ascertained by a colorimetric procedure with a standard process.

It is found in this respect that cytotoxicity of a significant level corresponding to growth inhibition of more than 30% is not present.

The invention is found to be of advantage in particular in performing cataract operations with dense cataracts and/or heavily pigmented fundi in which the fundus reflex is missing or is only slight. A good contrast is achieved between the colored anterior capsule and the subjacent tissue, by means of the dye agent.

### EXAMPLES

Embodiments by way of example of the dye agent in various buffer solutions are set forth hereinafter.

### Example 1

Patent blue V in a concentration of 1.2 g/l in a phosphate buffer solution.
200 ml of solution contain:
0.240 g of patent blue V
0.380 g of disodium hydrogen phosphate (Na₂HP0₄×2 H₂O)
0.060 g of sodium dihydrogen phosphate (NaH₂PO₄×2 H₂O)
1.640 g of sodium chloride (NaCl)
Sodium hydroxide for pH adjustment.

### Example 2

Patent blue V in a concentration of 1.2 g/l in a hydrogen carbonate buffer solution.
200 ml of solution contain:
0.240 g of patent blue V
0.420 g of sodium hydrogen carbonate (NaHCO₃)
1.640 g of sodium chloride (NaCl)
Sodium hydroxide for pH adjustment.

### Example 3

Patent blue V in a concentration of 1.2 g/l in a citrate buffer solution.
200 ml of solution contain:
0.240 g of patent blue V
0.216 g of trisodium citrate (C₆H₅Na₃O₇×2 H₂O)
1.640 g of sodium chloride (NaCl)
Sodium hydroxide for pH adjustment.

Identical embodiments in accordance with Examples 1, 2 and 3 can also be produced with brilliant blue R in a concentration of 1.2 g/l.

Preferably, in the case of the buffer solutions, the pH-value is adjusted by sodium hydroxide. It is however also possible for the solution itself to be adjusted to the desired pH value (neutral, slightly acid, slightly alkaline) within the preferred range of between 6.8 and 7.8. Adjustment of the concentration of patent blue of preferably between 0.6 and 2.5 g/l, in particular about 1.2 g/l, is effected by a suitable amount of patent blue V.

1-16. (canceled)

17. A method for staining cells in the human or animal body, the method comprising applying to the cells in the body a physiologically compatible aqueous solution of a dye which does not represent a vital dye and is biocompatible.

18. A method according to claim 17, which is for coloring, delimiting or separating membranes.

19. A method according to claim 18, which is for coloring, delimiting or separating membranes in the eye.

20. A method according to claim 18, which is for coloring, delimiting or separating membranes which are to be removed from an organ of the body,

21. A method according to claim 20, wherein the organ is the eye.

22. A method according to claim 16, which is for coloring the lens capsule of the eye.

23. A method according to claim 16, which is for coloring the lens anterior capsule in a cataract operation on the eye.

24. A method according to claim 16, which is for coloring membranes which have occurred due to disease in or at an organ of the body.

25. A method according to claim 24, wherein the organ is the retina of the eye.

26. A method according to claim 25, which is for coloring epiretinal membranes.

27. A method according to claim 26, which is for coloring a viscoelastic solution used in surgery.

28. A method according to claim 27, wherein the surgery is an ophthalmologic surgery.

29. A method according to claim 16, wherein the dye is dissolved in a neutral or weakly acid or weakly alkaline buffer.

30. A method according to claim 29, wherein the dye is dissolved in a buffer with a pH value of between about 6.8 and 7.8.

31. A method according to claim 30, wherein a phosphate, hydrogen carbonate or citrate buffer is used.

32. A method according to claim 16, wherein the dye is a triphenylmethane dye.

33. A method according to claim 32, wherein the concentration of the dye in the buffer solution is between about 0.3 and 2.5 g/l.

34. A method according to claim 33, wherein the concentration of the dye is about 1.2 g/l.

35. A method according to claim 32, wherein the dye is patent blue V.

36. A method according to claim 32, wherein the dye is brilliant blue R.

37. A physiologically compatible aqueous dye solution for coloring cells in the human or animal body, the dye solution comprising at least one dye which does not represent a vital dye and is biocompatible.

38. The dye solution of claim 37, wherein the dye is patent blue V or brilliant blue R.
